(19) United States
(12) Patent Application Publication
(10) Pub. No.: US 2025/0009926 A1
(43) Pub. Date: Jan. 9, 2025

(54) METHODS AND SYSTEMS FOR DISPENSING A VOLATILE MATERIAL

(71) Applicant: S. C. Johnson & Son, Inc., Racine, WI (US)

(72) Inventors: Joel E. Adair, Racine, WI (US); Sebastian D. Hasik, Antioch, IL (US)

(21) Appl. No.: 18/763,825

(22) Filed: Jul. 3, 2024

Related U.S. Application Data
(60) Provisional application No. 63/525,085, filed on Jul. 5, 2023.

(51) Int. Cl. A61L 9/03

(52) U.S. Cl. CPC A61L 9/037; A61L 2209/133

(57) ABSTRACT

A volatile material dispenser that includes a housing, a heater disposed within the housing, and a refill having a container with a volatile material disposed therein. A wick is in fluid communication with the volatile material and in thermal communication with the heater, which is a dual-section heating element that includes a first section and a second section that is spaced from the first section.

### CROSS REFERENCE TO RELATED APPLICATIONS

This application claims the benefit of and priority to U.S. Provisional Application Ser. No. 63/525,085, filed on Jul. 5, 2023, and entitled “METHODS AND SYSTEMS FOR DISPENSING A VOLATILE MATERIAL,” the entire content of which is incorporated by reference herein in its entirety and is considered a part of this application.

### BACKGROUND OF THE DISCLOSURE

### 1. Field of the Disclosure

The present disclosure relates generally to methods and systems for dispensing volatile materials, and more particularly, to systems and methods for volatizing a liquid containing a mixture of fragrances to achieve a desired volatile material character and intensity across a variety of use cases to improve a user's experience.

### 2. Description of the Background of the Disclosure

A multitude of volatile material diffusion devices or diffusers exist in the marketplace. Many of these devices are passive devices that require only ambient air flow to disperse the active liquid material therein. Other devices are battery-powered or receive household power via a plug extending from the device. Some known diffusers include a heating element for heating a volatile material to promote vaporization thereof. Other diffusers employ a fan or blower to generate air flow to direct volatile material out of the diffuser into the surrounding environment. Still other diffusers that dispense volatile materials utilize ultrasonic means to dispense the volatile materials therefrom. Fragrance compositions that are used in the aforementioned volatile material dispensers are composed of a mixture of volatile perfume raw materials, and it is not uncommon for a fragrance composition to be composed of over twenty different perfume raw materials. The chemical properties of perfume raw materials in a fragrance composition often vary widely in terms of polarity, density, vapor pressure, flash point, and other properties.

A problem with past volatile material dispensers is the inability to control both an intensity and fragrance character of the volatile material dispensed therefrom. While it is possible to emanate fragrance characteristics of a volatile material by applying different amounts of heat to isolate particular fragrance characteristics, an increase in applied heat necessarily results in a higher intensity of volatile that is dispensed to the surrounding environment. As a result, methods and systems for controlling both an intensity and fragrance character of a volatile material are useful.

### SUMMARY OF THE DISCLOSURE

In some embodiments, a volatile material dispenser includes a housing that defines a chimney aperture, a heater disposed within the housing, and a refill having a container with a volatile material disposed therein. A wick is in fluid communication with the volatile material and in thermal communication with the heater. An airflow enhancement mechanism is disposed within the housing. The heater is configured to heat the volatile material to a first temperature to achieve a first fragrance characteristic and is further configured to heat the volatile material to a second temperature to achieve a second fragrance characteristic that is different than the first fragrance characteristic. The airflow enhancement mechanism is configured to achieve a first fragrance intensity at a first setting and is further configured to achieve a second fragrance intensity at a second setting that is different than the first setting.

In some embodiments, the heater is a first heater, and the airflow enhancement mechanism includes a second heater. In some embodiments, the second heater is movable and the first heater is a static heater. In some embodiments the heater includes a third heater. In some embodiments, the first temperature and the second temperature are at least 30° C. apart. In some embodiments, the airflow enhancement mechanism includes a wick sheath. In some embodiments, the airflow enhancement mechanism includes a mechanical output adjuster. In some embodiments, the airflow enhancement mechanism includes a chimney sheath. In some embodiments, the heater has a cooler portion and a warmer portion. In some embodiments, the heater is configured such that the cooler portion is larger than the warmer portion. In some embodiments, the wick is movable closer to and farther from the warmer portion and the cooler portion. In some embodiments, the heater is configured to collapse and expand when moving farther from and closer to the wick, respectively.

In some embodiments, a volatile material dispenser includes a housing, a heater disposed within the housing, and a refill having a container with a volatile material disposed therein. The refill includes a wick that is in fluid communication with the volatile material and that is in thermal communication with the heater. In some embodiments, the heater of the volatile material dispenser is a dual-section heating element that includes a first section and a second section that is spaced from the first section. In some embodiments, the heater and the wick have an elliptic cross section. In some embodiments, the first section of the heater is a warm section and the second section of the heater is a cool section. In some embodiments, the warm section of the heater is smaller than the cool section of the heater. In some embodiments, the dispenser includes a first configuration in which the wick is disposed near the warm section of the heater and the dispenser outputs a first fragrance characteristic and a second configuration in which the wick is disposed near the cool section of the heater and the dispenser outputs a second fragrance characteristic.

In some embodiments, the dispenser outputs a similar intensity of the first fragrance characteristic when the dispenser is in the first configuration as it does the second fragrance characteristic when the dispenser is in the second configuration. In some embodiments, the wick is configured to be moveable to allow the dispenser to transition between the first configuration and the second configuration. In some embodiments, the heater is configured to be moveable to allow the dispenser to transition between the first configuration and the second configuration. In some embodiments, a temperature of the warm section of the heater and a temperature of the cool section of the heater are at least 30° C. apart. In some embodiments, the temperature of the warm section of the heater and the temperature of the cool section of the heater are at least 60° C. apart.

In some embodiments, a method of operating a volatile material dispenser includes the step of providing a volatile material dispenser that includes an air enhancement mechanism, a heater disposed within a housing that produces a convection zone, and a refill having a wick in fluid communication with a volatile material disposed within a container. The method of operating a volatile material dispenser may also include the steps of adjusting the temperature of the heater to adjust a fragrance characteristic that is released from the evaporated volatile material during an operation cycle of the volatile material dispenser and adjusting the air flow into the convection zone by using the air enhancement mechanism to control an intensity of the fragrance characteristic that is released during the operation cycle from the volatile material dispenser. Further, the air enhancement mechanism used for adjusting the air flow into the convection zone may be a cylindrical chimney sheath that defines a height and includes at least a first air port and a second air port that each extend therethrough and define heights. In some embodiments, a bridge of material may be disposed on opposing sides of each air port such that each bridge extends between the first air port and the second air port. In some embodiments, the height of at least one of the air ports is between about 5% and about 20% of the chimney sheath height. In some embodiments, the height of at least one of the air ports is between about 30% and about 50% of the chimney sheath height. In some embodiments at least one air port is disposed entirely within a bottom 50% of the chimney sheath height. In some embodiments, the first air port and the second air port are disposed on opposing sides of the chimney sheath. In some embodiments, the first air port and the second air port of the chimney sheath are rectangular.

In some aspects, a volatile material dispenser includes a housing, a heater disposed within the housing, and a refill. The refill includes a container with a volatile material disposed therein and a wick that is in fluid communication with the volatile material and that is in thermal communication with the heater. Further, the heater is configured to collapse when moving closer to the wick and expand when moving farther from the wick. In some embodiments, the dispenser includes an airflow enhancement mechanism in the form of a chimney sheath. In some embodiments, the heater is in the form of a tri-sectional heating element. In some embodiments, the sectional heating elements are connected with a wire and are in direct contact when in a fully collapsed configuration. In some embodiments, the heater in the fully collapsed configuration is oriented above the wick.

### BRIEF DESCRIPTION OF THE DRAWINGS

FIG. 1 is an isometric view of a volatile material dispenser as disclosed herein;

FIG. 2 is an exploded view of the volatile material dispenser of FIG. 1;

FIG. 3 is an enlarged, cross-sectional view of an upper portion of the volatile material dispenser of FIG. 1;

FIG. 4 is a bottom view of the volatile material dispenser of FIG. 1 showing the dispenser without the refill and with an air flow conductor in a full open configuration;

FIG. 5 is a bottom view of the volatile material dispenser of FIG. 1 showing the dispenser without the refill and with the air flow conductor in a partially open configuration;

FIG. 6 is a bottom view of the volatile material dispenser of FIG. 1 showing the dispenser without the refill and with the air flow conductor in a full closed configuration;

FIG. 7 is a bar graph showing various output rates of the volatile material dispenser of FIG. 1, which are dependent upon applied heat and air flow conductor configuration;

FIG. 8 is an exploded view of a volatile material dispenser with another air flow conductor in the form of a chimney sheath along with various chimney sheath alternatives;

FIG. 9 is a rear, cross-sectional view of another volatile material dispenser with a moveable wick sheath showing the wick sheath in a vertically down configuration;

FIG. 10 is a rear, cross-sectional view of the volatile material dispenser of FIG. 9 showing the wick sheath in a vertically up configuration;

FIG. 11 is a bar graph showing various output rates of the volatile material dispenser of FIG. 9, which are dependent upon applied heat and wick sheath configuration;

FIG. 12 is a rear, cross-sectional view of another volatile material dispenser with a moveable heater shown in a vertically up configuration;

FIG. 13 is a rear, cross-sectional view of the volatile material dispenser of FIG. 12 showing the heater in a vertically down configuration;

FIG. 14 is a rear, cross-sectional view of yet another volatile material dispenser with a dual-section heating element showing a wick tilted toward a smaller, warmer portion of the heating element;

FIG. 15 is a rear, cross-sectional view of the volatile material dispenser of FIG. 14 showing the wick tilted toward a larger, cooler section of the heating element;

FIG. 16 is a schematic top view of an elliptical dual-section heating element showing a wick positioned closer to a smaller, warmer section of the heating element;

FIG. 17 is a schematic top view of a circular dual-section heating element showing a wick positioned closer to a smaller, warmer section of the heating element;

FIG. 18 is a schematic side view of a dual-section heating element showing a variable height of the heater with multiple configurations of a wick shown in dashed lines;

FIG. 19 is a rear, cross-sectional view of still another volatile material dispenser with a static heating element and a moveable heating element, with the moveable heating element in a vertically up configuration;

FIG. 20 is a rear, cross-sectional view of the volatile material dispenser of FIG. 19, with the moveable heating element in a vertically down configuration;

FIG. 21 is a rear, cross-sectional view of another volatile material dispenser with low, medium, and high output heating elements;

FIG. 22 is a rear, cross-sectional view of yet another volatile material dispenser with a heating element attached to a motorized adjustment mechanism, with the heating element in a vertically up configuration;

FIG. 23 is a rear, cross-sectional view of the volatile material dispenser of FIG. 22, with the heating element in a vertically down configuration;

FIG. 24 is a rear, cross-sectional view of another volatile material dispenser with a tri-sectional heating element shown in a vertically up, collapsed configuration;

FIG. 25 is a top schematic view of the tri-sectional heating element and wick of FIG. 24 shown in the collapsed configuration;

FIG. 26 is a rear, cross-sectional view of the volatile material dispenser of FIG. 24, with the tri-sectional heating element shown in a vertically down, expanded configuration;

FIG. 27 is a top schematic view of the tri-sectional heating element and wick of FIG. 24 shown in the expanded configuration; and

FIG. 28 is a right, cross-sectional view of another volatile material dispenser with a volatile material heating element and an air preheating heating element.

### DETAILED DESCRIPTION

The present disclosure relates to volatile material dispensing systems and methods that provide for controlling fragrance characteristics and the intensity of a volatile material being dispensed to the surrounding atmosphere to offer more customizability and to enhance a user's experience. By regulating the heat and airflow applied to a volatile material, the concentration and fragrance characteristics of the volatile material can be controlled and adjusted to be consistent based on a desired user experience. The systems and methods disclosed herein allow the volatile material dispensing system to control a temperature applied by the heating element, which modifies the fragrance characteristic of the volatile, and to further control the dispersion rate of the volatile material by controlling airflow around a wick. Control of the dispersion rate can be based upon the location or disposition of the heater, the temperature and output of the heater(s), the number of heaters, the airflow surrounding the wick, and the exposed surface area of the wick.

By controlling the temperature applied by the heating element(s) in a targeted fashion, the volatile material dispenser can adjust or modify the volatile material evaporation rate and fragrance characteristics depending upon the desired fragrance characteristic(s) and intensity. Thus, the volatile material dispenser may be configured to achieve four or more settings that allow for different fragrance characteristics and intensity. While the various examples described herein relate to only four different configurations based on the combination of two different fragrance characteristics and two different intensities, it is contemplated that any number of fragrance characteristics and any number of intensities may be achieved through the systems and methods described herein. Throughout the disclosure, the terms “about” and “approximately” refer to a range of values±5% of the numeric value that each term precedes. As noted herein, all ranges disclosed within this application are inclusive of the outer bounds of the range.

The term “fragrance,” as used herein, refers to any substance or a mixture of substances such as a perfume designed to emit an aromatic scent. A wide variety of chemicals are known for fragrance (i.e., perfume) uses, including materials such as aldehydes, ketones, and esters. More commonly, naturally occurring plant and animal oils and exudates comprising complex mixtures of various chemical components are known for use as fragrances. The present disclosure relates to volatile material dispensers that are configured to emit any fragrance or combinations of fragrances. Many fragrances comprise a number of different perfume raw materials, each having their own chemical characteristics or properties, which generally vary in terms of polarity, density, vapor pressure, flash point, and other properties.

The fragrances and other volatile compositions of the present application may comprise a single chemical or may comprise a sophisticated complex mixture of natural and synthetic chemical components, all chosen to provide any desired odor or effect. For example, the fragrances and perfumes of the present application may comprise one or more perfume raw materials. The term “perfume raw materials,” as used herein, refers to any compound or substance that is useful in imparting an odor, fragrance, essence, or scent either alone or in combination with other “perfume raw materials.” Mixtures of perfume raw materials are known by those skilled in the art of fragrances and perfumes as “accords.” The term “accord,” as used herein, refers to a mixture of two or more perfume raw materials which are artfully combined to impart a scent, odor, essence, or fragrance characteristic.

To that end, a first fragrance may be identified that has a fragrance characteristic that is achieved within the first temperature range, a second fragrance may be identified that has a fragrance characteristic that is achieved within the second temperature range, and a third fragrance may be identified that has a fragrance characteristic that is achieved within the third temperature range. Additionally, the first fragrance may have a peak or desired first fragrance characteristic at a first temperature, the second fragrance characteristic may have a peak or desired second fragrance characteristic that is different than the first fragrance characteristic at a second temperature, and the third fragrance may have a peak or desired third fragrance characteristic that is different than the first and second fragrance characteristics at a third temperature. The volatile material dispensers disclosed herein are configured to heat volatile materials to specific temperatures to achieve targeted fragrance characteristics. In some examples, a difference of at least 20° C., or at least 30° C., or at least 40° C., or at least 50° C. is required between temperatures associated with the first, second, or third fragrance characteristics.

The following embodiments of the present disclosure generally relate to methods and systems for adjusting fragrance characteristics and intensity by modifying one or more of three distinct variables: 1) an amount of airflow, 2) an amount of exposed surface area of a wick, and 3) a temperature and location of the heater. By modifying one or more of the three variables listed above, it has been found that intensity adjustability and character shift adjustability are possible to achieve in a targeted fashion. In one particular example, adjusting a heater to a “high” setting may result in a cinnamon flavor being perceived by a user, and adjusting the heater to a “low” setting may result in an apple flavor. Absent other variable changes, higher heat (temperature) applied to a wick generally results in a first fragrance characteristic and lower heat (temperature) applied to the wick generally results in a second fragrance characteristic, different than the first fragrance characteristic. As a result, the below embodiments help to achieve distinct fragrance characteristics at different intensities by adjusting the heat applied by a heater while simultaneously adjusting one or more airflow enhancement mechanisms, as described below.

In some embodiments, the fragrance characteristics are associated with peak fragrance intensities of the respective compositions, or only some of the fragrance characteristics are associated with peak fragrance intensities. Still further, in some embodiments, the fragrance characteristic may be associated with a non-fragranced active, such as a pest control active, which may be volatized at a higher rate than other compositions within the refill. In such an embodiment, the fragrance characteristics are reflective of an attribute or characteristic of the composition that is non-fragrance based. The increase in evaporated volatile material may be accomplished without saturating the surrounding environment with evaporated volatile material by utilizing an airflow enhancement mechanism. The airflow enhancement mechanisms disclosed herein are configured to facilitate or modify airflow surrounding the wick, which may include modifying the surrounding air temperature and/or adjusting an exposed surface area of the wick.

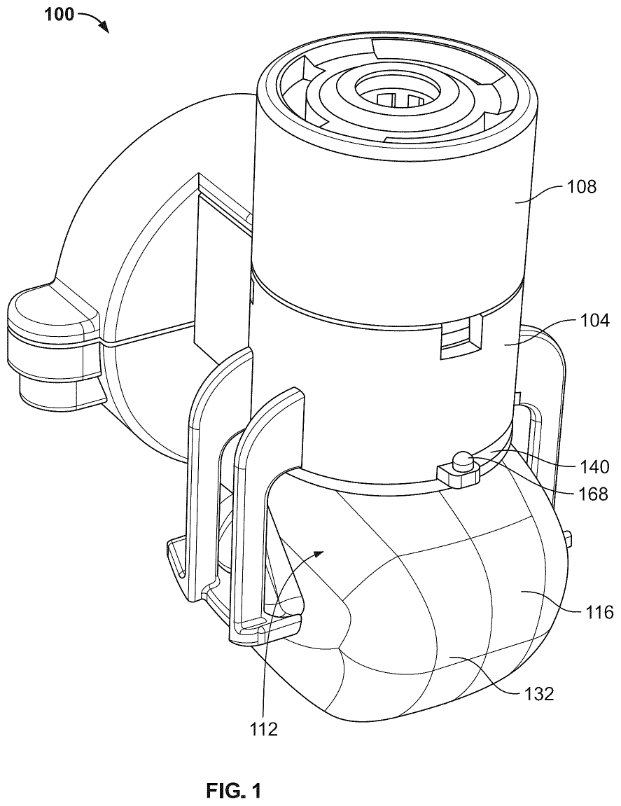

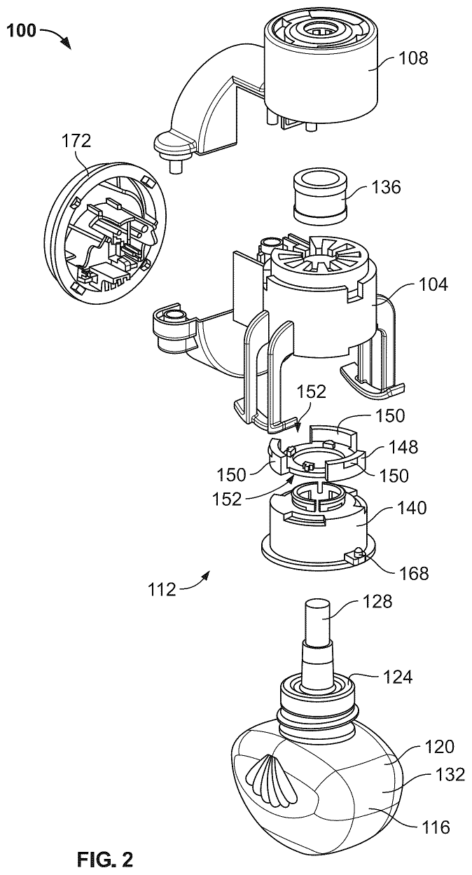

Referring now to the figures, FIGS. 1-6 depict a first embodiment of a volatile material dispenser 100 in accordance with the present disclosure. Referring specifically to FIGS. 1 and 2, the volatile material dispenser 100 includes a first or lower housing 104, a second or upper housing 108, an airflow enhancement mechanism 112 in the form of an air flow conductor, and a refill 116. As illustrated in FIG. 2, the refill 116 includes a container 120, a plug assembly or lid 124, and a fluid delivery system 128 in the form of a wick that extends through the lid 124. The wick 128 is in fluid communication with a volatile material 132 and in thermal communication with a heater 136. The air flow conductor 112 includes a mechanical output adjuster 140 and an upper vent ring 148, which are used in combination to achieve a desired air flow of air surrounding the wick 128 of the refill 116. The upper vent ring 148 is a ring that has a set of three protrusions 150 that are spaced radially around the ring 148. In between the protrusions 150 are three radially spaced voids 152. The refill 116 includes the volatile material 132 disposed within the container 120. The first housing 104 is configured to receive the air flow conductor 112 and is further configured to house the heater 136, which may be movable. The heater 136 is in thermal communication with the volatile material 132 through the wick 128. The volatile material dispenser 100 may be configured to control the volatile material concentration in the environment by controlling one or more of airflow, heat applied to the wick, the temperature of the heat, and the exposed surface area of the wick. The volatile material 132 within the refill 116 is in fluid communication with the heater 136.

Still referring to FIG. 2, the volatile material 132 may be in direct contact with the heater 136 through the wick 128. In some embodiments the wick 128 is not in direct contact with the heater 136. In some embodiments, the refill 116 is in thermal communication with the heater 136 such that the wick 128 brings the volatile material 132 close enough in proximity to the heater 136 to facilitate evaporation of the volatile material 132 faster than an evaporation in a control setting. In some embodiments, the heater 136 is in communication with the refill 116 via the wick 128, which transports the volatile material 132 from the container 120 to the heater 136. In some embodiments, the wick 128 may be a sintered wick such as a POREX® Wick. In some embodiments, the fluid delivery system shown as the wick 128 in FIG. 2 is another, different type of liquid transfer mechanism. In some embodiments, the fluid delivery system 128 may be a gravity fed injector or an apparatus for storing fluid that has a connection to a drip pipe disposed below the apparatus.

In some embodiments, the fragrance characteristics and volatile material characteristics can be enhanced through different durations of operation and/or different frequencies of operation. For example, different frequencies of operation could include the volatile material dispenser 100 operating a duty cycle every 5 minutes or every 15 minutes. In some embodiments, the fragrance characteristics and volatile material characteristics can be enhanced through an algorithm that varies the temperature of activation in a predetermined fashion and/or through the use of an on/off timer. In some embodiments, the varying of a speed of the airflow enhancement mechanism 112 when the heater 136 is turned “on” is used to enhance the characteristics of the evaporated volatile material 132. Controlling the temperature of the heater 136 and rate of air displacement may result in controlling evaporated volatile material 132 intensities and fragrance characteristics of the evaporated volatile materials 132, which creates a consistent volatile material intensity and fragrance experience despite the particular user environment. The operation of the volatile material dispenser 100 may be adjusted directly through user manipulated controls.

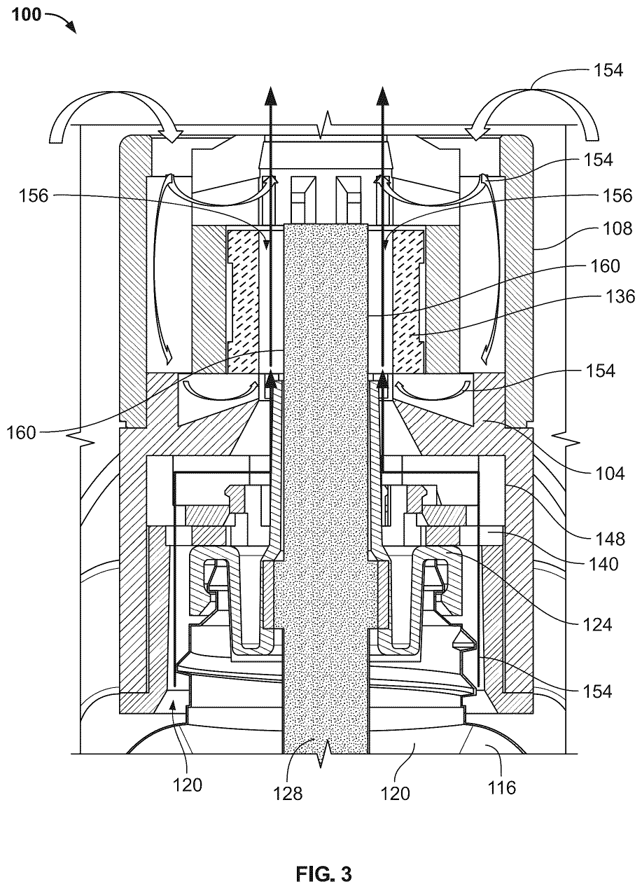

As illustrated in FIG. 3, the first housing 104 and the second housing 108 define multiple airflow paths, denoted by arrows 154, which allow air to enter into a convection zone 156 or oven, that surrounds an exposed region 160 of the wick 128. The heater 136 is shown being in indirect contact with the exposed region 160 of the wick 128, and is disposed within the convection zone 156. The air flow conductor 112 is also shown in FIG. 3, which is adjustable to allow more or less air to enter into and out of the convection zone 156. Further, the heater 136 is movable vertically up and down, which allows the heater 136 to be in direct contact with more or less of the exposed region 160 of the wick 128. This movement also allows for more or less of the exposed region 160 of the wick 128 to be in direct contact with the heater 136. As noted above, the fragrance characteristic and intensity can be adjusted by one or both of moving the heater 136 and by adjusting the air flow conductor 112. More specifically, adjusting a temperature of the heater 136 can modify the fragrance characteristic, while moving the heater 136 and adjusting the air flow conductor 112 can modify the intensity of the fragrance since modifying the airflow and exposed surface of the wick 128 can result in a change in fragrance intensity.

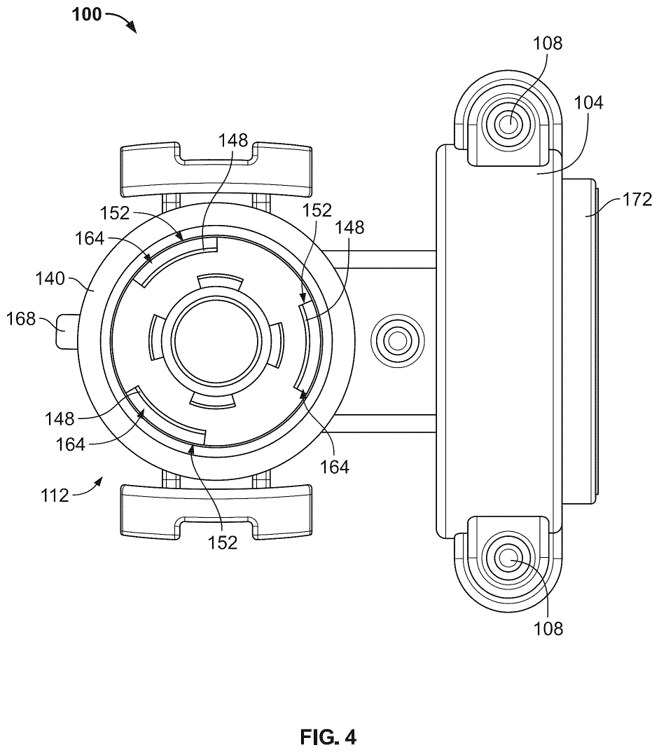

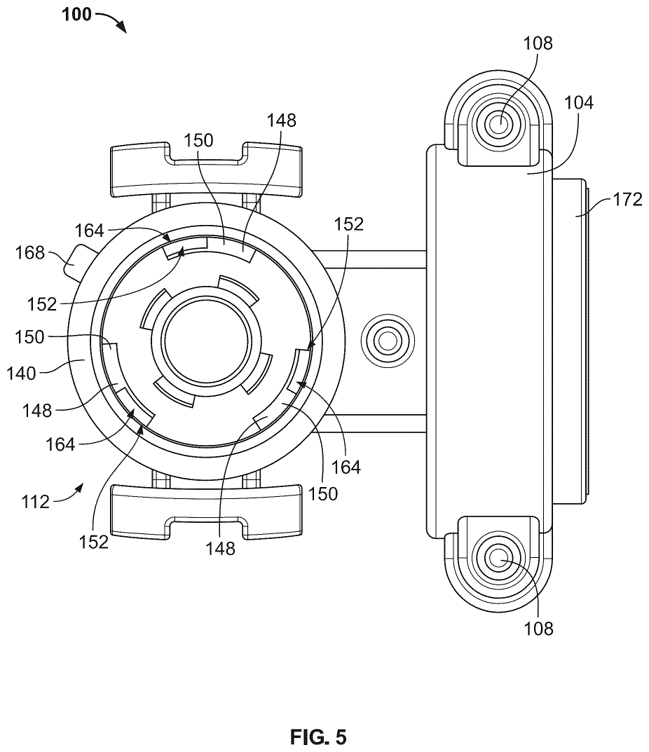

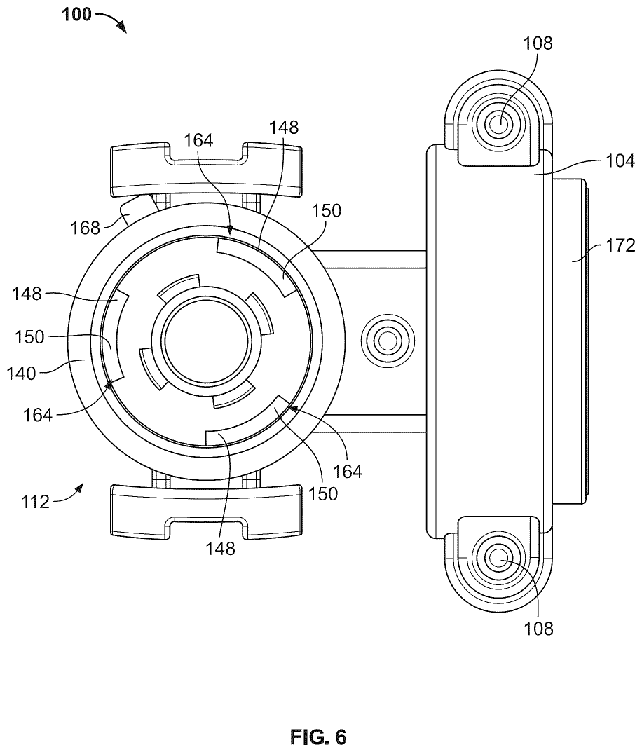

Referring to FIGS. 4-6, the volatile material dispenser 100 of FIG. 1 is shown with the refill 116 having been removed for clarity. Referring to FIG. 4, the air flow conductor 112 is in a full open configuration where a set of three apertures 164, which are spaced radially along and through the mechanical output adjuster 140, align completely with voids 152 of the upper vent ring 148 to create additional pathways for air to flow into the convection zone 156. When the apertures 164 are aligned completely with the voids 152 of the upper vent ring 148, a maximum amount of airflow can occur through the apertures 164 to the convection zone 156. FIG. 5 illustrates an intermediate configuration where the air flow conductor 112 is in an about half-open or half-closed configuration. In the half-closed configuration, about half of each aperture 164 is positioned such that it abuts the protrusions 150 of the upper vent ring 148 that acts to limit the size of the pathway for air to flow through to the convection zone 156, which means that an intermediate (˜50%) amount of airflow can occur through the convection zone 156. Finally, FIG. 6 illustrates a full closed configuration where each aperture 164 in its entirety abuts the protrusions 150 of the upper vent ring 148. As a result, there is limited to no pathway for air to travel through the apertures 164, which means that a minimum amount of airflow can occur through the convection zone 156. Each of the configurations shown in FIGS. 4-6 impacts the airflow through the convection zone 156, and each of the configurations can further be modified by adjusting a vertical placement of the heater 136.

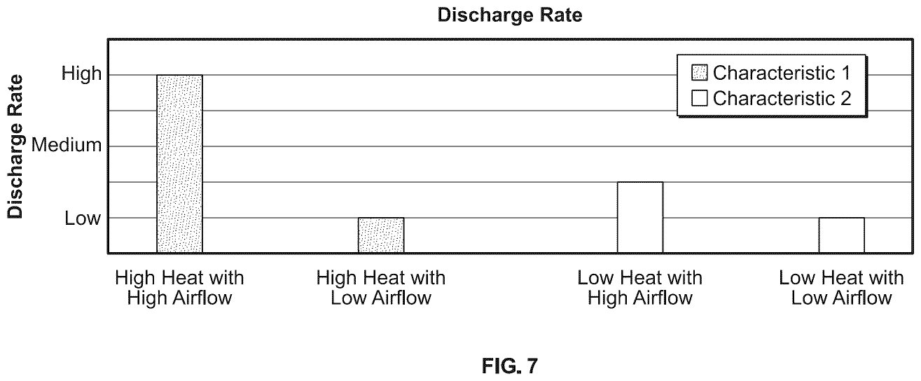

For example, and referring to FIG. 7, different discharge rates can be achieved based on manipulating two of the three variables noted above, namely, adjusting a temperature of the heater 136 and adjusting the airflow into the convection zone 156. As shown in the bar graph, applying a “high” level of heat with a “high” level of airflow results in a first fragrance characteristic with a high discharge rate. Further, applying a “high” level of heat with a “low” level of airflow results in the first fragrance characteristic with a low discharge rate. Still further, applying a “low” level of heat with a “high” level of airflow results in a second fragrance characteristic with a medium-low discharge rate. Finally, applying a “low” level of heat with a “low” level of airflow results in the second fragrance characteristic with a low discharge rate.

The intensity or discharge rates shown in FIG. 7 can be further modified by moving the heater 136 up or down such that more or less of the exposed region 160 of the wick 128 is exposed to the convection zone 156. While the heater 136 of the present embodiment may be used to modify how much of the exposed region 160 of the wick 128 is exposed to the convection zone 156, some of the embodiments below include non-heating elements, such as a sheath, that impact how much of the exposed region 160 of the wick 128 is exposed to the convection zone 156. Rather than adjusting the proximity of the heater 136 to the wick 128, some heating elements are configured to achieve different fragrance characteristics by modifying an amount of heat applied by the heater 136. In some embodiments, 4.0 Watts (W) of power may be associated with a “low” setting, 5.0 W of power may be associated with a “medium” setting, and 6.0 W of power may be associated with a “high” setting.

Referring again to FIGS. 1-6, the refill 116 may include a pre-dosed pad or gel. In some embodiments, the refill 116 may be integral and nonremovable from the volatile material dispenser 100. In some embodiments, the refill 116 may be a refillable container that may include a cartridge. The cartridge may be configured to include additional elements, such as a base that is coupled with the cartridge. In some embodiments, the refill 116 may be configured to be removeable from the volatile material dispenser 100. In some embodiments, the refill 116 is configured for one-time use such that the wick 128 and the heater 136 are embedded within the refill 116. In some embodiments, the refill 116 may be configured to be used for multiple uses and may also be detachable from the volatile material dispenser 100.

In some embodiments, the refill 116 holds the volatile material 132 that may include one or more compositions, which may be any suitable liquid or liquids, and may include one or more active ingredients. Active ingredients include, but are not limited to, one or more of a cleaner, an insecticide, an insect repellant, an insect attractant, a disinfectant, a mold or mildew inhibitor, an antimicrobial, a fragrance comprised of one or more aroma chemicals, a disinfectant, an air purifier, an aromatherapy scent, an antiseptic, an odor eliminator, a positive fragrancing active material, an air-freshener, a deodorizer, a medicinal component, an inhalant (e.g., for relieving a cough or congestion), or the like, and combinations thereof. The volatile material dispenser 100 disclosed herein may be used as a pest control product that has the ability to operate in both an unfragranced and a fragranced repellent mode (e.g., at a lower temperature setting which outputs a very light or negligible fragrance and at a higher temperature setting which outputs a fragrance).

Still referring to FIGS. 1-6, the airflow enhancement mechanism 112 is a mechanical device that is rotated by a user grasping a handle 168, which extends radially outward from the mechanical output adjuster 140. In the present embodiment, the mechanical output adjuster 140 may be rotated to achieve an infinite number of positions between the maximum open and closed configurations. However, in alternative embodiments, the mechanical output adjuster 140 may be manipulable between discrete configurations, for instance, a first configuration, a second configuration, and a third configuration. As discussed in detail below, the airflow enhancement mechanism 112 may alternatively or additionally include a fan, a wick sheath, or one or more additional heaters that aid in achieving a desired airflow within or around the volatile material dispenser 100 and the wick 128.

To that end, the airflow enhancement mechanism 112 may be a fan at the base of the volatile material dispenser 100 that blows the evaporated volatile material from the wick 128 through and out of the volatile material dispenser 100. In some embodiments and as discussed in detail below, the airflow enhancement mechanism 112 may be a secondary heating element (not shown) that creates a convection tube within the volatile material dispenser 100 that carries the evaporated volatile material from the refill 116 away from the volatile material dispenser 100. In some embodiments, the airflow enhancement mechanism 112 may include internal or external structure that is configured to take advantage of naturally occurring diffusion or the surrounding air flow in the environment to provide enhanced air flow and/or mitigate power consumption of the volatile material dispenser 100. In some embodiments, the airflow enhancement mechanism 112 is an air pump or a pneumatic pump.

Alternatively, the airflow enhancement mechanism 112 may be a venturi tube with a secondary heating element (not shown) separate from the heater 136. The venturi tube may be configured to create a pressure differential that may promote air movement and function as the airflow enhancement mechanism 112. In some embodiments, the airflow enhancement mechanism 112 is a ceiling fan wherein the volatile material dispenser 100 is mounted on one of the fans blades. In some embodiments, the airflow enhancement mechanism 112 may be a diaphragm, a vacuum pump, or a compressor. In some embodiments, the airflow enhancement mechanism 112 is an external apparatus that moves and has the volatile material dispenser 100 attached to the external apparatus. The external apparatus that moves may be a car, a bike, a scooter, a dog, a treadmill, a stationary bike, a door, a window, or any other object that moves.

To that end, the airflow enhancement mechanism 112 may be a vehicle air vent and the volatile material dispenser 100 may be configured to be mounted to the vehicle air vent. In some embodiments, the airflow enhancement mechanism 112 may be configured to have an adjustable nozzle that is designed to influence the exit of the evaporated volatile material into the surrounding environment. In such embodiments, the nozzle may be configured to increase or decrease the exit angle to influence the dispersion of the evaporated volatile material into the surrounding environment. In some embodiments, the airflow enhancement mechanism 112 uses ionic wave generation. In some embodiments, the volatile material dispenser 100 may not include an active airflow enhancement mechanism 112 and may instead only include a passive airflow enhancement mechanism 112, such as the mechanical output adjuster 140. Thus, the volatile material dispenser 100 may be a passive dispenser and configured to disperse amongst the environment through ordinary diffusion and/or other environmental forces. In some embodiments, the airflow enhancement mechanism 112 may be configured to push air through a hose which enters an emission cavity within the refill 116 and mixes with the evaporated volatized material within the tank of the refill 116.

Still referring to FIGS. 1-6, the power supply 172 may be configured to receive a USB—C type plug, that can be used to charge or power the volatile material dispenser 100. In some embodiments, the volatile material dispenser 100 may receive power from a wall outlet, a car lighter socket, or another source of power as the power supply 172. In some embodiments the power supply 172 may be a battery which could include a rechargeable battery, a one-time use battery, a lead-acid battery, a nickel-cadmium battery, a nickel-metal hybrid battery, a lithium-ion battery, an alkaline battery, a zinc-carbon battery, a coin cell battery, a zinc-air battery, a sealed lead-acid battery, or any other device known in the art that holds energy in the form of chemicals. In some embodiments, the power supply 172 may use a combination of battery types and/or the power supply 172 may be a combination of power sources such as a battery and a wall charger. In some embodiments, the power supply 172 may implement power conditioning to transform the line voltage to 5V DC. In some embodiments, the power supply 172 may not implement power conditioning.

In some embodiments, the heater 136 may be a ceramic annular disk with an inlayed potted metal oxide resistor. In some embodiments, the heater 136 comprises ceramic with an inlayed potted metal oxide resistor that is not in an annular shape. Further, the heater 136 may be a tubular metal oxide resistor, a kapton heater, a foil heater, or a copper heating coil. In some embodiments, the heater 136 comprises a honeycomb configuration such that it is considered a honeycomb heater. Further, the heater 136 may be configured to receive the wick 128 that extends upwardly into contact with the heater 136. As noted above, the heater 136 may be manipulable, e.g., vertically, to cover more or less of the exposed region 160 of the wick 128, which can impact an intensity of the fragrance. Alternatively, the heater 136 may be a contactless laser LED or a consumable heating element such as a tungsten filament. In some embodiments, the heater 136 may be integrated in the refill 116. In some embodiments, the heater 136 comprises a plurality of heating elements that surround the wick 128. The plurality of heating elements may be arranged in such a way that they are able to sequentially heat the wick 128 or may heat the wick with different temperatures based on a desired fragrance characteristic or intensity. In some embodiments, the heater 136 may be a nichrome wire that is embedded in the wick 128 that is attached to and/or along portions of the container 120 of the refill 116. In some embodiments, the heater 136 may be a pin-point heater. In some embodiments, the heater 136 may comprise one or more resistors.

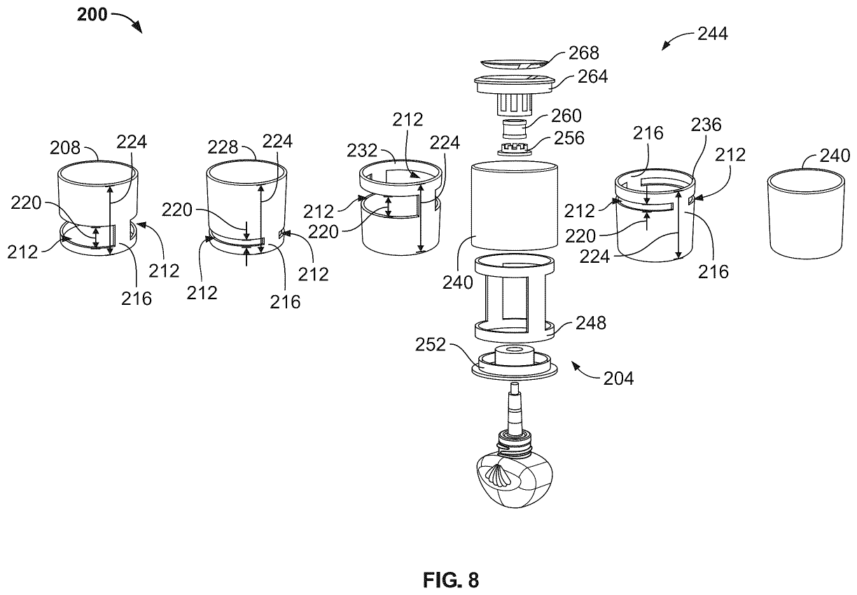

FIG. 8 is an exploded view of a volatile material dispenser 200 with another airflow enhancement mechanism 204 in the form of a chimney sheath along with various sheath alternatives. For example, a first sheath 208 is shown having a plurality of air ports 212 that are disposed on opposing sides thereof, which are rectangularly shaped and include bridges 216 of material that extend therebetween. The air ports 212 of the first sheath 208 comprise an air port height 220 that is between about 30% and about 50% of a total height 224 of the first sheath 208. Further, the air ports 212 are disposed entirely within a bottom 50% of the total height 224 of the first sheath 208. A second sheath 228 is also shown having a plurality of the air ports 212 disposed on opposing sides thereof. The air ports 212 of the second sheath 228 are also rectangularly shaped and include bridges 216 of material that extend therebetween. The air ports 212 of the second sheath 228 comprise an air port height 220 that is between about 5% and about 20% of a total height 224 of the second sheath 228. Further, the air ports 212 are disposed entirely within a bottom 25% of the total height 224 of the second sheath 228.

Still referring to FIG. 8, a third sheath 232 is shown having a plurality of air ports 212 that are disposed on opposing sides thereof and include bridges 216 of material that extend therebetween. The air ports 212 of the third sheath 232 comprise an air port height 220 that is between about 20% and about 40% of the total height 224 of the third sheath 232. Further, the air ports 212 are disposed entirely within a top 50% of the total height 224 of the third sheath 232. A fourth sheath 236 is shown, which also includes a plurality of air ports 212 that are disposed on opposing sides thereof and separated by bridges 216. The air ports 212 of the fourth sheath 236 comprise an air port height 220 that is between about 10% and about 25% of a total height 224 of the fourth sheath. Further, the air ports 212 are located entirely within an upper 30% of the fourth sheath 236. Finally, a fifth sheath 240 is shown, which does not include any air ports 212. All of the sheaths 208, 228, 232, 236, 240 may be configured to operate with an adjustment assembly 244 that further includes an internal ring housing 248, a lower cap 252, a heater stand 256, a heater 260, an upper cap 264, and a top member 268.

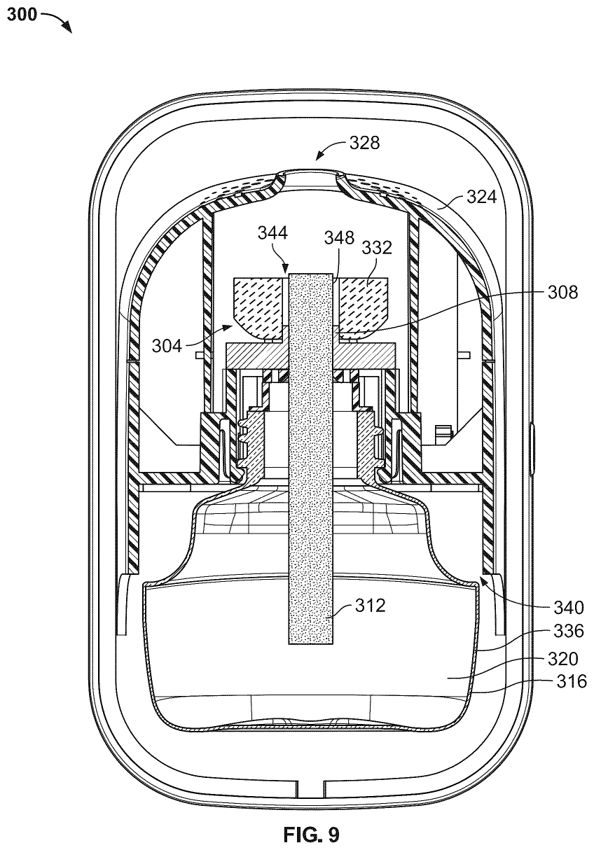

FIG. 9 is a rear, cross-sectional view of another volatile material dispenser 300 with yet another airflow enhancement mechanism 304 in the form of a moveable wick sheath 308, which is shown in a vertically “down” configuration. The wick sheath 308 may comprise a thermally insulating material that is configured to move vertically to expose more or less of a wick 312 of a refill 316, which impacts a release rate of a volatile material 320, i.e., more wick exposed means more material is released. The volatile material dispenser 300 further includes a housing 324 defining an upper chimney aperture 328, a heater 332, and the wick sheath 308 that is configured to surround the wick 312 of the refill 316. The refill 316 further includes a container 336 that contains the volatile material 320, which is in fluid communication with the wick 312. A lower airflow gap 340 defined between the refill 316 and the housing 324 allows air to flow into a convection zone 344 caused by the heater 332, and volatized air flows out of the upper chimney aperture 328. In the present embodiment, the heater 332 and the wick sheath 308 are both movable, thus, a fragrance character shift is caused by adjustment of a temperature of the heater 332, and movement of the wick sheath 308 to expose more or less of the wick 312 impacts an intensity release of the volatile material.

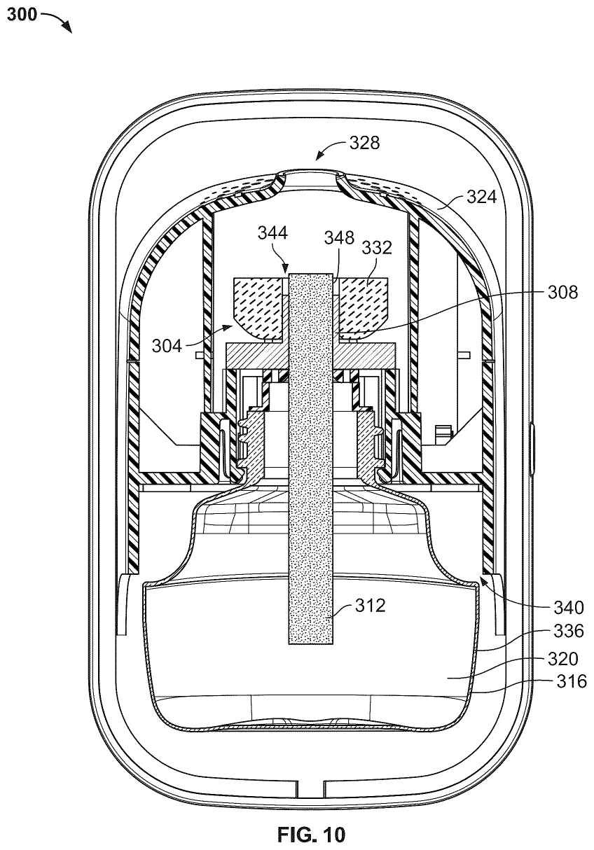

Referring to FIG. 10, the wick sheath 308 is shown in a vertically “up” configuration, which means that an exposed region 348 of the wick 312 has been reduced. With a smaller surface area of the wick 312 exposed to the convection zone 344, an intensity of release of the volatile in the “up” configuration of FIG. 10 is less than an intensity of release of the volatile in the “down” configuration of FIG. 9. It should be appreciated that while “up” and “down” are used to refer to the particular configurations, the configurations could instead be considered “first” and “second” configurations, or the wick sheath 308 may be reversed such that the more-exposed configuration is the “up” configuration and the less-exposed configuration is the “down” configuration. This applies to all of the embodiments disclosed herein.

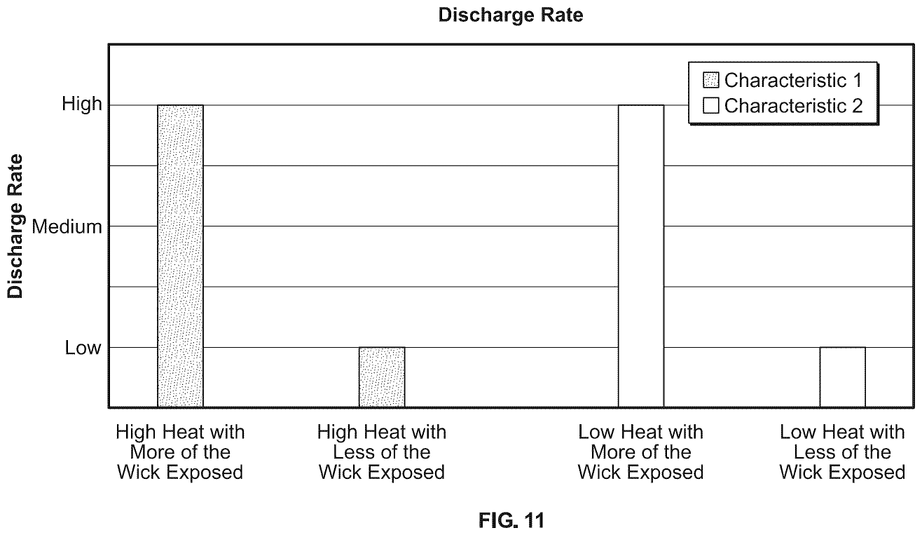

FIG. 11 illustrates a bar chart showing the comparison of fragrance characteristics based on applied heat and whether more or less of the wick 312 is exposed to the convection zone 344. To that end, applying high heat with more of the wick 312 exposed results in a higher intensity of the first fragrance characteristic, and applying high heat with less of the wick 312 exposed results in a lower intensity of the first fragrance characteristic. Further, applying low heat with more of the wick 312 exposed results in a higher intensity of the second fragrance characteristic, and applying low heat with less of the wick 312 exposed results in a lower fragrance intensity of the second fragrance characteristic.

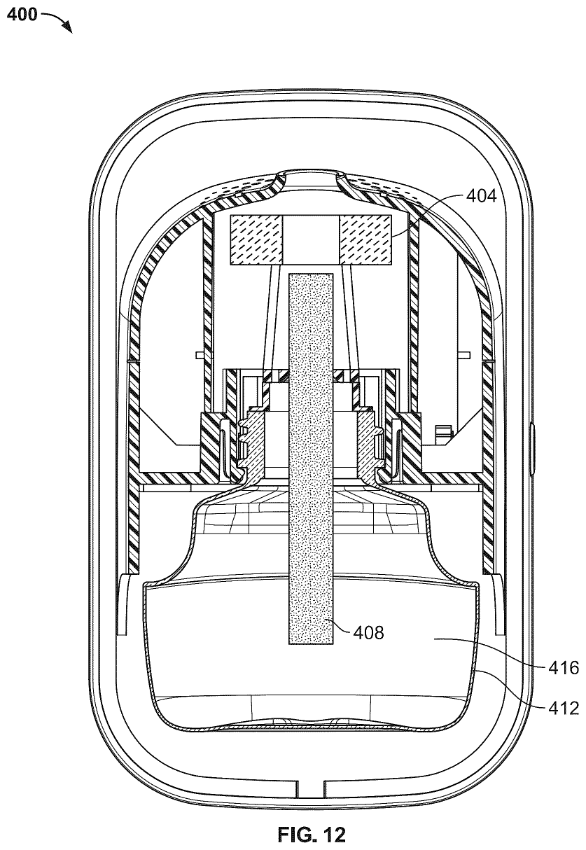

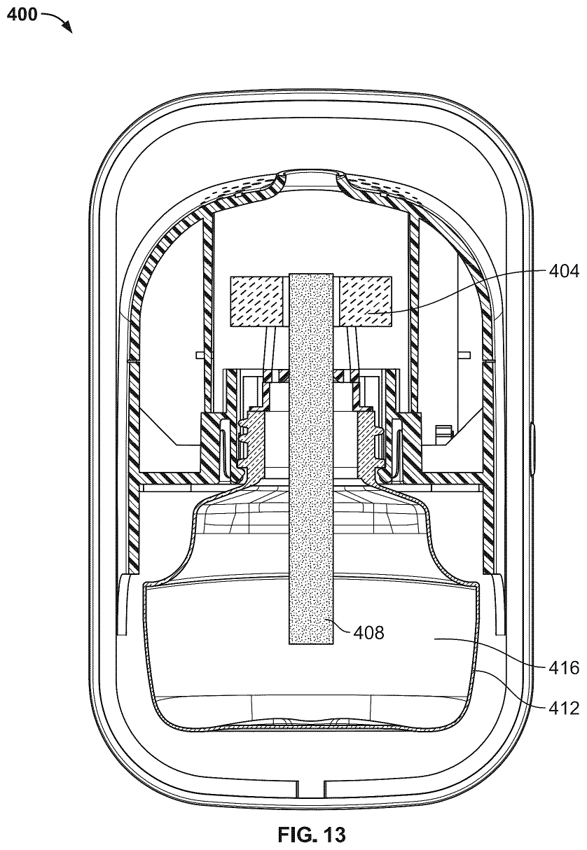

FIGS. 12 and 13 are rear, cross-sectional views of another volatile material dispenser 400 with a heater 404 that is movable. FIG. 12 shows the heater 404 in a vertically up configuration, and FIG. 13 illustrates the heater 404 in a vertically down configuration. In some embodiments, the heater 404 is movable by adjusting a switch or lever (not shown) that is configured to be manipulated by a user. By adjusting the switch or lever, a user can move the heater 404 closer to or farther from a wick 408. A container 412 with a volatile material 416 is also shown in FIGS. 12 and 13. In some embodiments, as the heater 404 moves toward the wick 408, the wattage of and heat applied by the heater 404 decreases, and as the heater 404 moves away from the wick 408, the wattage of and heat applied by the heater 404 increases. As a result, by applying a lower amount of heat closer to the wick 408, as shown in FIG. 13, a different fragrance characteristic is emitted than applying a higher amount of heat farther from the wick 408, as shown in FIG. 12. One or more of the airflow enhancement mechanisms disclosed herein may also be combined with the movable heater of FIGS. 12 and 13.

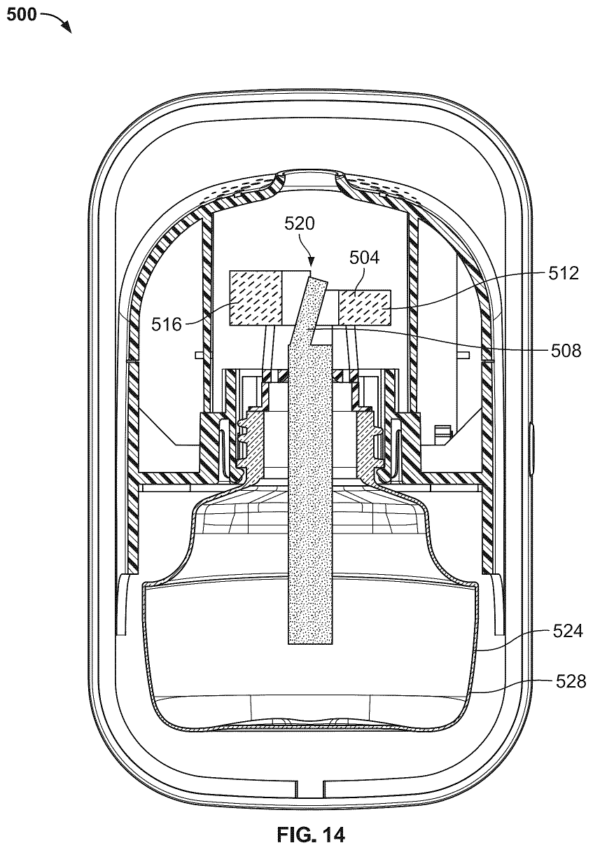

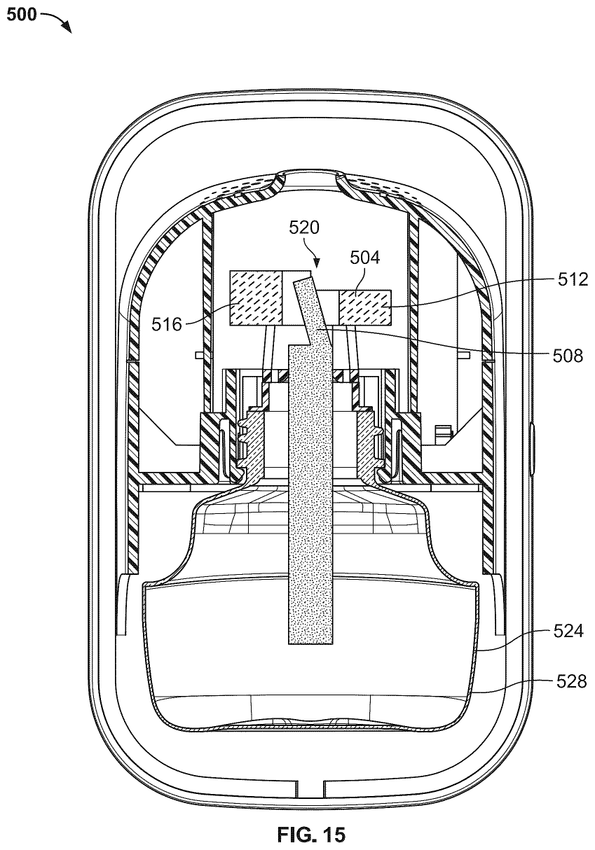

Now referring to FIGS. 14 and 15, rear, cross-sectional views are shown of yet another volatile material dispenser 500 with a heater 504 that is a dual-section heating element. In FIG. 14, a wick 508 is tilted toward a smaller, warmer portion 512 of the heater 504, while FIG. 15 illustrates the wick 508 being tilted toward a larger, cooler portion 516 of the heater 504. Since the heater 504 of FIGS. 14 and 15 is not symmetric and has an irregular shape, different temperatures can be delivered to different portions or regions of the wick 508. For example, tilting the wick 508 toward the cooler portion 516 produces an output at scent characteristic A, and tilting the wick 508 toward the warmer portion 512 would heat a smaller portion of the wick 508 at a higher temperature, resulting in a similar or identical output at scent characteristic B. In some embodiments, the difference in temperature between the cooler portion 516 and the warmer portion 512 of the heater 504 is between 30° C. and 60° C. In some embodiments, the heater 504 may have a circular footprint when viewed from above, i.e., looking down at the wick 508, but a conical shape when viewed in elevation. The volatile material dispenser 500 of FIGS. 14 and 15 thus results in two heating profiles/characteristics having constant output since the wick 508 is centered between the cooler portion 516 and the warmer portion 512 of the heater 504. As illustrated in FIGS. 14 and 15, an upper end 520 of the wick 508 may have a smaller diameter than the portion of the wick 508 that is disposed within a container 524 of a refill 528.

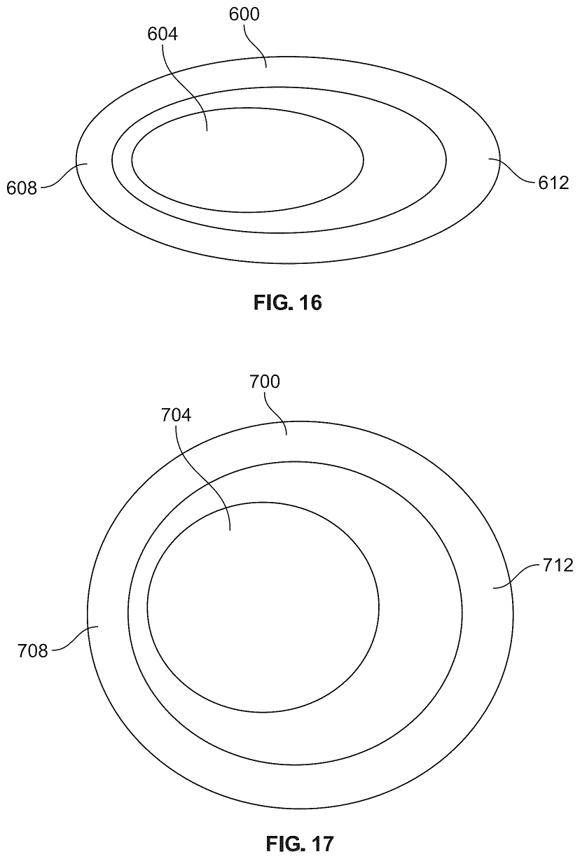

FIGS. 16 and 17 illustrate schematic top views of two different configurations for a dual-section heating element, similar to the heater 504. FIG. 16 illustrates a heater 600 that is a dual-section heating element with a wick 604 having an elliptical cross section, and the heater 600 having an elliptical cross section. The heater 600 further includes a warmer portion 608 and a cooler portion 612. FIG. 17 illustrates a heater 700 that is a dual-section heating element with a wick 704 having a circular profile, and the heater 700 having a circular profile. The heater 700 of FIG. 17 also includes a warmer portion 708 and a cooler portion 712. The heaters 600, 700 and wicks 604, 704 of FIGS. 16 and 17 both illustrate the wicks 604, 704 being positioned closer to the warmer portions 608, 708 of the heaters 600, 700. In some embodiments, the wicks 604, 704 are movable from being closer to the warmer portions 608, 708 to being closer to the cooler portions 612, 712, and in some embodiments, the heaters 600, 700 are movable or rotatable to adjust which of the warmer portions 608, 708 and the cooler portions 612, 712 are closer to the wicks 604, 704.

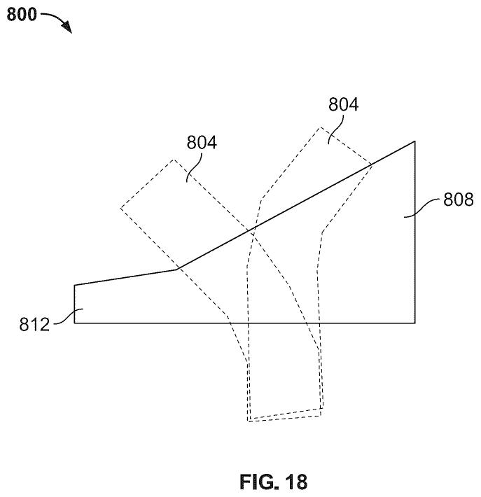

Referring now to FIG. 18, a schematic side view a heater 800 that is a dual section heater is shown with multiple configurations of a wick 804 shown in dashed lines. As shown, the heater 800 has a variable height. The heater 800 of FIG. 18 further includes a cooler portion 808 and a warmer portion 812 of the heater 800, similar to the heaters 504, 600, 700 discussed above with respect to FIGS. 14-17. In the present embodiment, the cooler portion 808 of the heater 800 is larger and taller than the warmer portion 812 of the heater 800, although in some embodiments the opposite may be true. By allowing for physical adjustment of one or both of the wick 804 and the heater 800 for exposure to different portions of the heater 800, i.e., the warmer portion 812 and the cooler portion 808, both the fragrance character and the intensity of the fragrance can be controlled. In that sense, the heater 800 may be configured to achieve a certain temperature in various locations thereof, but the positioning of the heater 800 with respect to the wick 804 allows for intensity control of the fragrance to the surrounding environment.

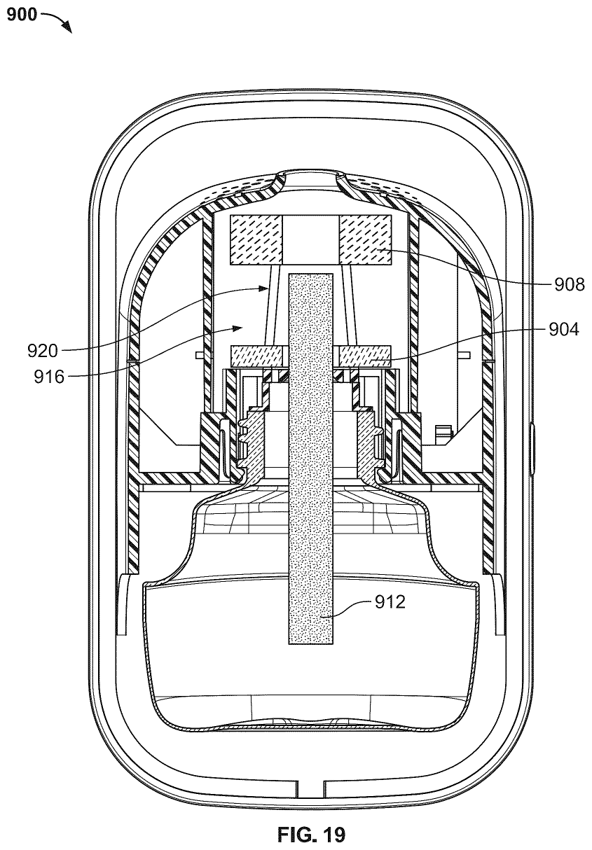

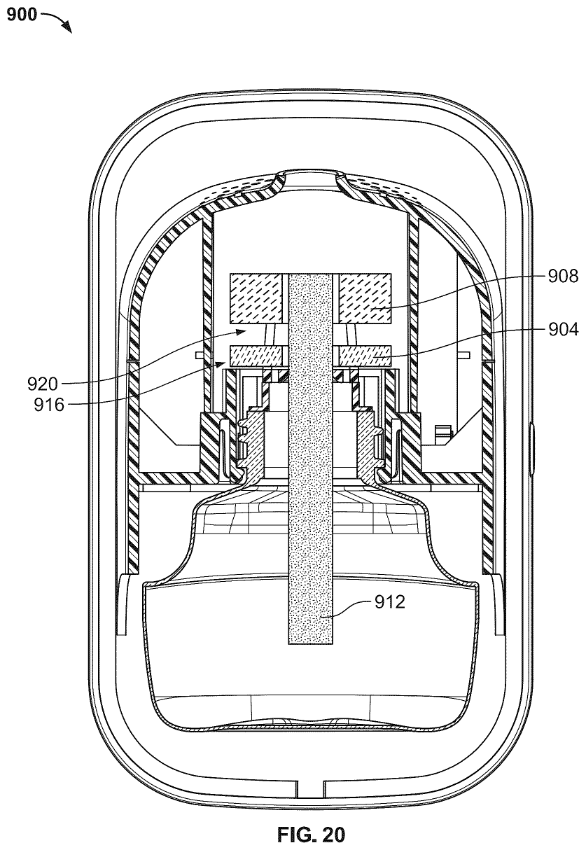

Referring now to FIGS. 19 and 20, yet another volatile material dispenser 900 is shown, which includes a heater 904 in the form of a static heater that remains in place, and a movable heater 908 that is configured for movement closer to or farther from a wick 912. In particular, FIG. 19 illustrates the movable heater 908 in a vertically up configuration, farther from the wick 912, and FIG. 20 illustrates the moveable heater 908 in a vertically down configuration, closer to the wick 912. In the present embodiment, the moveable heater 908 may be considered an airflow enhancement mechanism 916 since the location of the moveable heater 908 results in different convection rates and airflow within the volatile material dispenser 900. In some embodiments, the heater 904 is always configured to be “on” at a low setting around the wick 912, and the moveable heater 908 is movable up and down to influence the fragrance characteristics by adding additional heat into a convection zone 920 at various locations. For example, by moving the heater 908 closer to the wick 912, an increased temperature will be achieved within the convection zone 920, with an increased release rate and a first fragrance characteristic. By moving the heater 908 away from the wick 912, a decreased temperature will result, as well as a lower release rate and different fragrance characteristic.

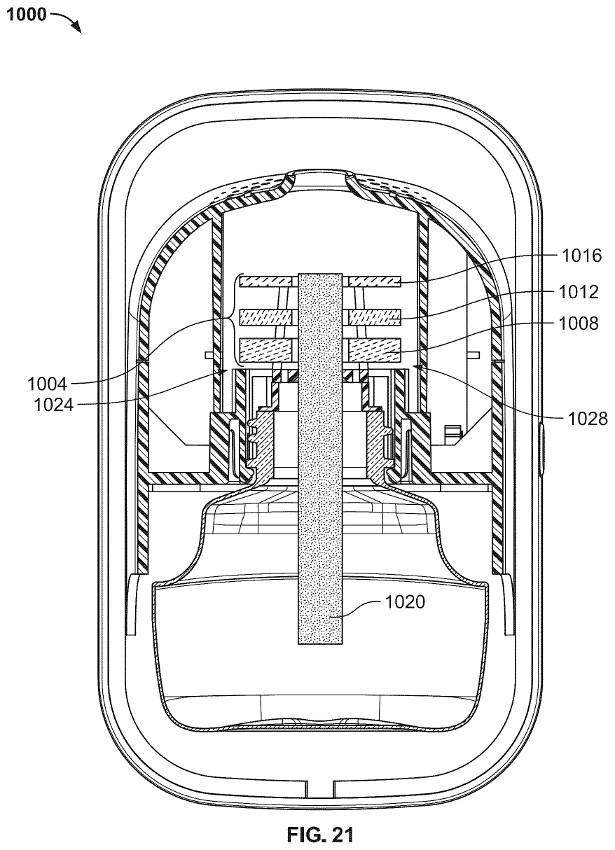

Referring now to FIG. 21, a rear, cross-sectional view of another volatile material dispenser 1000 is illustrated, which includes a heating element 1004 in the form of a first heater 1008, a second heater 1012, and a third heater 1016. The heaters 1008, 1012, 1016 are configured as a low temperature heater, a medium temperature heater, and a high temperature heater, respectively. Each of the heaters 1008, 1012, 1016 are configured such that if only one heater is on, the fragrance release rate/intensity will be the same. The first heater 1008 is the largest, and exposes the largest surface area of a wick 1020 to heat. The second heater 1012 is the next largest, but exposes less surface area of the wick 1020 to heat. The third heater 1016 is the smallest, and exposes a smaller surface area of the wick 1020 to heat. The heaters 1008, 1012, 1016 may be configured to produce three distinct characteristics, and various different settings may be configurable to produce the different fragrance characteristics.

For example, Setting 1 may be configured to only apply wattage to the first heater 1008, Setting 2 may be configured to only apply wattage to the second heater 1012, Setting 3 may be configured to only apply wattage to the third heater 1016, Setting 4 may be configured to apply wattage to both the first heater 1008 and the second heater 1012, and Setting 5 may be configured to apply wattage to all of the heaters 1008, 1012, 1016. The different Settings may be configured to achieve different fragrance characteristics, and given that more heat is applied from Setting 1 to Setting 5, the intensity of the fragrance increases moving from Setting 1 to Setting 5. Finally, any of the heaters 1008, 1012, 1016 may be considered an airflow enhancement mechanism 1024 since additional heat applied by any one of the heaters 1008, 1012, 1016 may cause different airflow into a convection zone 1028.

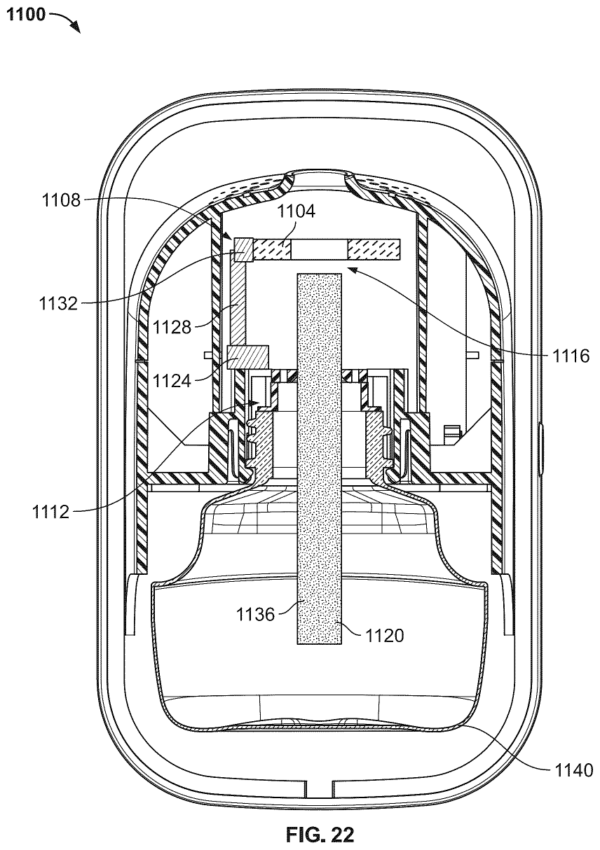

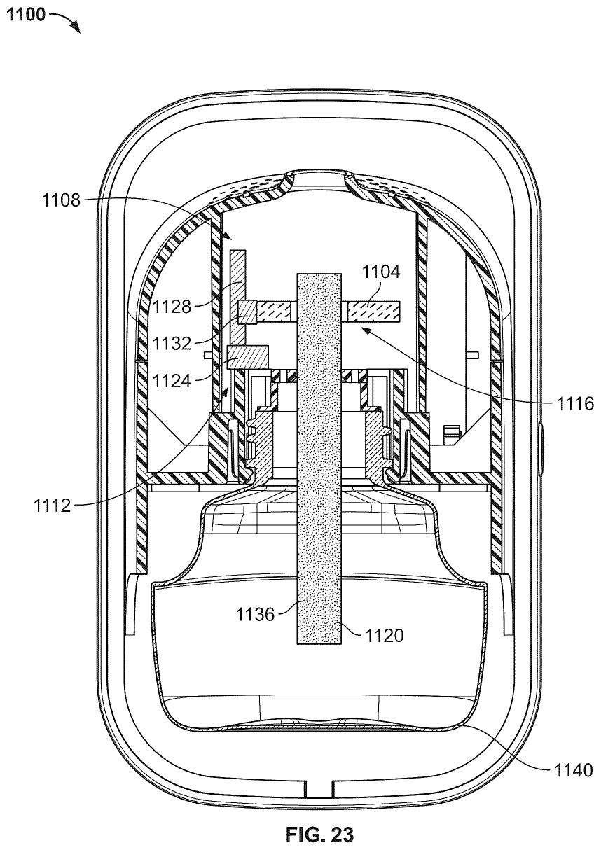

Referring now to FIG. 22, a rear, cross-sectional view of yet another volatile material dispenser 1100 is shown, which includes a heater 1104 in the form of a movable heater that is coupled with a motorized adjustment mechanism 1108, with the heater 1104 shown in a vertically up configuration. The motorized adjustment mechanism 1108 may be considered an airflow enhancement mechanism 1112 in the present embodiment since a location of the heater 1104 within the volatile material dispenser 1100 modifies the airflow into the convection zone 1116. To that end, the heater 1104 is movable toward and away from a wick 1120 using a motor 1124. The motor 1124 is coupled with a shaft 1128, which is further coupled with chassis 1132 that is attached to the heater 1104. In the present embodiment, the motor 1124 drives the shaft 1128, which is engaged with a gear on the heater 1104 or on the chassis 1132, to constantly or periodically move the heater 1104 relative to the wick 1120. Both the fragrance characteristic and the release rate of the fragrance change when the heater 1104 is moved closer to and farther from the wick 1120. FIG. 23 is a rear, cross-sectional view of the volatile material dispenser of FIG. 22, with the heating element in a vertically down configuration.

The volatile material dispenser 1100 may be configured to move the heater 1104 closer to or farther from a volatile material 1136 disposed within the wick 1120 depending on an intensity selection of the user. In such embodiments, the volatile material dispenser 1100 may be configured to move the heater 1104 closer to a refill 1140 to increase the intensity of the heat transferred from the heater 1104 to the refill 1140. Thus, the amount of the volatile material 1136 evaporated into the environment may increase as a result of an increased rate in energy transfer from the heater 1104 to the volatile material contained within the refill 1140. In contrast, the volatile material dispenser 1100 may be configured to move the heater 1104 away from the refill 1140 to decrease the amount and intensity of the energy transferred from the heater 1104 to the refill 1140. Thus, the amount and intensity of the volatile material released into the environment may decrease. In some embodiments, the volatile material dispenser 1100 may be configured to control the rate of volatile material evaporation by moving the heater 1104 along the refill 1140, which may comprise a pre-dosed pad or gel, to continuously expose the heater 1104 to a new area or region of the volatile material composition. In some embodiments, the heater 1104 may be moved using a pendulum or another type of oscillating device.

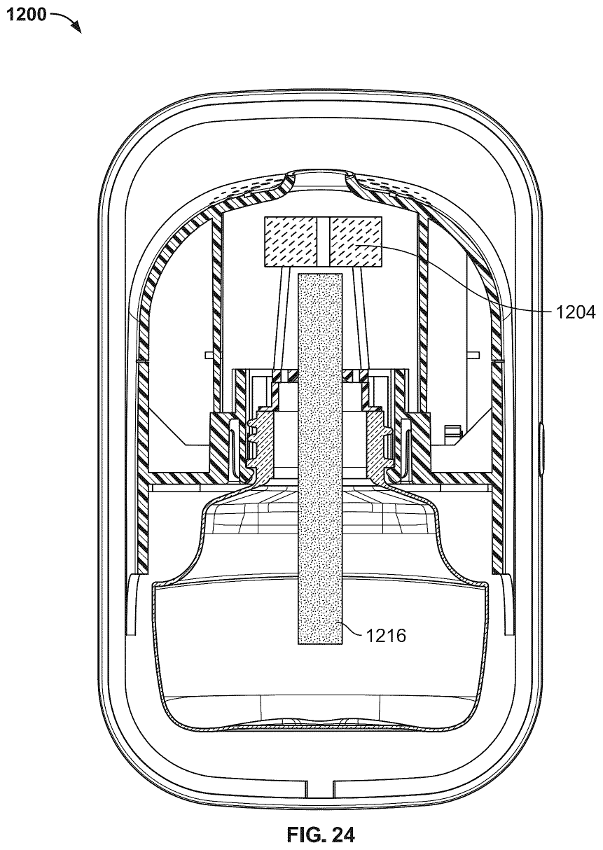

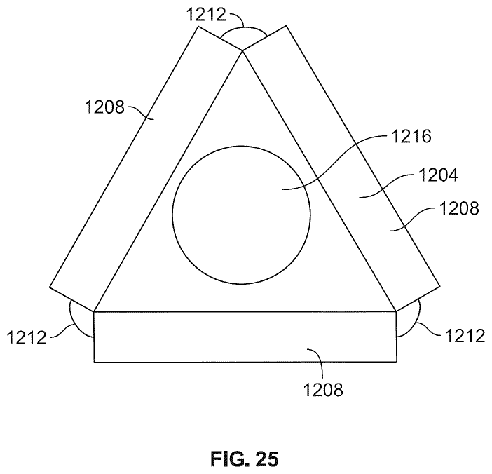

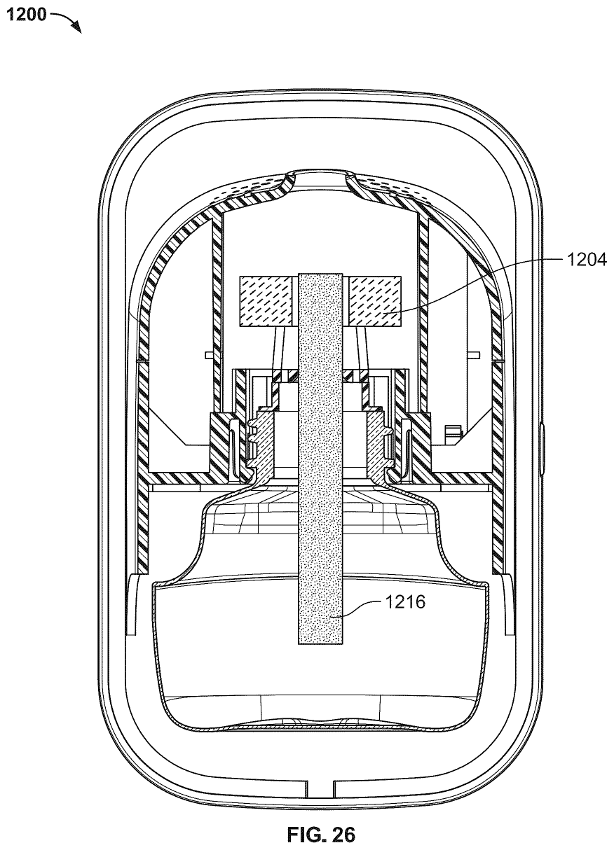

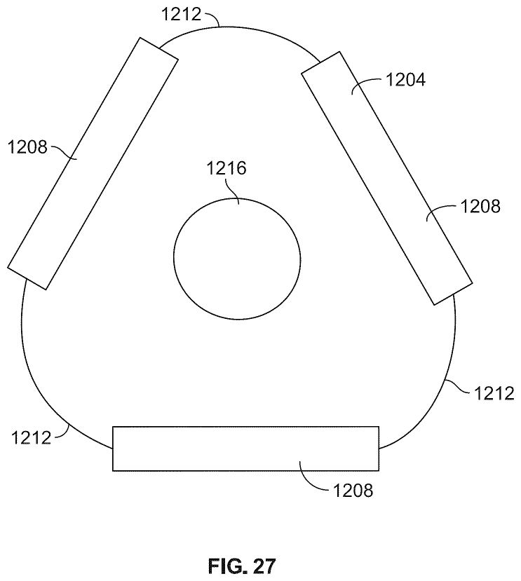

Referring now to FIGS. 24-27, yet another volatile material dispenser 1200 is shown, which includes a heater 1204 in the form of a tri-sectional heating element, which is shown in a vertically up, collapsed configuration in FIG. 24. In the present embodiment, the heater is broken up into three different segments 1208 that are configured to be moved to a position nearest the wick (see FIG. 26) where they are all touching (or close to it) and a position away from the wick where they are spaced apart (i.e., some radial gaps). Referring to FIG. 25, the segments 1208 may be coupled to one another with a wire 1212. Comparing FIGS. 24 and 26, when the heater 1204 moves from the vertically up configuration to the vertically down configuration, the segments 1208 become spaced apart such that they surround a wick 1216. As the heater segments 1208 move upward, i.e., moving to the configuration of FIG. 24, the segments 1208 move closer together. As shown in FIG. 27, the heater segments 1208 move away from one another as they come closer to the wick 1216. The segments 1208 may be movable up and down through any of the heater movement devices disclosed herein. Further, the volatile material dispenser 1200 may include airflow enhancement mechanisms disclosed herein.

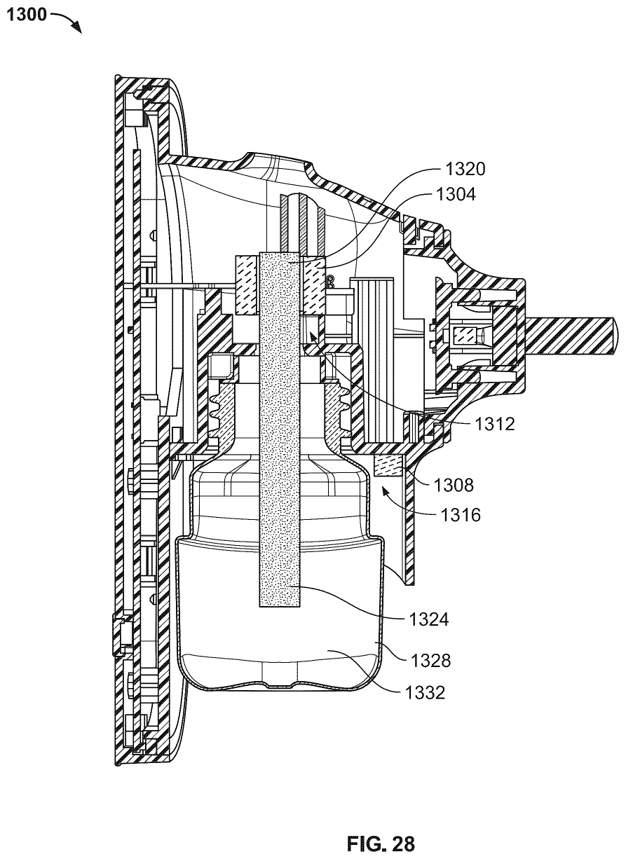

Referring now to FIG. 28, still another volatile material dispenser 1300 is shown, which includes a heater 1304 and an airflow or secondary heater 1308, which is disposed beneath the convection zone 1312, and is configured to draw air into the volatile material dispenser 1300. In the illustrated embodiment, the airflow heater 1308 is considered an airflow enhancement mechanism 1316 since it is configured to draw air into the volatile material dispenser 1300. The heater 1304 may be movable up and down, as described above, and the airflow heater 1308 may be configured to allow for the adjustability of the air being drawn into the volatile material dispenser 1300. For example, the airflow heater 1308 may be adjustable to increase a temperature of the air being supplied to the exposed region 1320 of a wick 1324 to encourage increased output. In some embodiments, a damper or adjustable air flow mechanism, such as a fan, may be utilized to adjust the airflow into the convection zone 1312 surrounding the wick 1324.

Still referring to FIG. 28, the flow rate of a volatile material 1328 from a refill 1332 to the heater 1304 through the wick 1324 may be controlled to control the amount of the volatile material 1328 that is evaporated and released into the environment. In some embodiments, the flow rate of volatile material 1328 from the refill 1332 to the heater 1304 can be controlled using the secondary heater 1308 to heat or cool the refill 1332. Heating the refill 1332 with the secondary heater 1308 may decrease the viscosity of the volatile material 1328, thereby increasing the flow from the refill 1332 to the heater 1304 via the wick 1324. In a similar fashion, the secondary heater 1308 may be turned off or the power output may be lowered to increase the viscosity of the volatile material 1328 in the refill 1332 to reduce the flow rate of the volatile material 1328 from the refill 1332 to the heater 1304 via the wick 1324.

In any of the embodiments described above, the volatile material dispensers may use duty cycles to achieve the desired volatile material concentration in the surrounding environment. The volatile material dispensers may be configured to have a power switch that is used to power “on” and “off” the volatile material dispenser. When the power switch is activated to an “on” position, the controller can direct electric current to flow to the heater and the airflow enhancement mechanism using power supplied by the power supply.

The devices or systems disclosed herein can be used, manufactured, or installed using methods embodying aspects of the disclosure. Correspondingly, any description herein of particular features, capabilities, or intended uses of a device or system is generally intended to include disclosure of a method of using such devices for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated, discussion herein of any method of manufacturing or use for a particular device or system, including installing the device or system, is intended to inherently include disclosure, as embodiments of the disclosure, of the utilized features and configurations, and implemented capabilities of such device or system. Further, any of the heaters, the wicks, airflow displacement mechanisms, or refills, of any of the embodiments may be combined with those of any other embodiment(s).

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Any of the embodiments described herein may be modified to include any of the structures or methodologies disclosed in connection with different embodiments. Further, the present disclosure is not limited to the dispensing systems of the type specifically shown. Still further, the methods and systems of any of the embodiments disclosed herein may be modified to work with any type of volatile material dispenser.

### INDUSTRIAL APPLICABILITY

Numerous modifications to the present disclosure will be apparent to those skilled in the art in view of the foregoing description. Accordingly, this description is to be construed as illustrative only and is presented for the purpose of enabling those skilled in the art to make and use the disclosure. The exclusive rights to all modifications which come within the scope of the appended claims are reserved.

We claim:

1. A volatile material dispenser, comprising:
a housing;
a heater disposed within the housing; and
a refill having a container with a volatile material disposed therein and a wick in fluid communication with the volatile material and in thermal communication with the heater,
wherein the heater is a dual-section heating element that includes a first section and a second section that is spaced from the first section.

2. The volatile material dispenser of claim 1, wherein the heater and the wick have an elliptic cross section.

3. The volatile material dispenser of claim 1, wherein the first section of the heater is a warm section and the second section of the heater is a cool section.

4. The volatile material dispenser of claim 3, wherein the warm section of the heater is smaller than the cool section of the heater.

5. The volatile material dispenser of claim 4, wherein the dispenser includes a first configuration in which the wick is disposed near the warm section of the heater and the dispenser outputs a first fragrance characteristic and a second configuration in which the wick is disposed near the cool section of the heater and the dispenser outputs a second fragrance characteristic, and
wherein the dispenser outputs a similar intensity of the first fragrance characteristic when the dispenser is in the first configuration as it does the second fragrance characteristic when the dispenser is in the second configuration.

6. The volatile material dispenser of claim 5, wherein the wick is configured to be moveable to allow the dispenser to transition between the first configuration and the second configuration.

7. The volatile material dispenser of claim 5, wherein the heater is configured to be moveable to allow the dispenser to transition between the first configuration and the second configuration.

8. The volatile material dispenser of claim 5, wherein a temperature of the warm section of the heater and a temperature of the cool section of the heater are at least 30° C. apart.

9. The volatile material dispenser of claim 8, wherein the temperature of the warm section of the heater and the temperature of the cool section of the heater are at least 60° C. apart.

10. A method of operating a volatile material dispenser comprising the steps of:
providing a volatile material dispenser that includes an air enhancement mechanism, a heater disposed within a housing that produces a convection zone, and a refill having a wick in fluid communication with a volatile material disposed within a container;
adjusting a temperature of the heater to adjust a fragrance characteristic that is released from evaporated volatile material during an operation cycle of the volatile material dispenser; and
adjusting the air flow into the convection zone by using the air enhancement mechanism to control an intensity of the fragrance characteristic that is released during the operation cycle of the volatile material dispenser,
wherein the air enhancement mechanism used for adjusting the air flow into the convection zone is a cylindrical chimney sheath that defines a height and includes at least a first air port and a second air port that each extend therethrough and define heights, and
wherein a bridge of material is disposed on opposing sides of each air port such that each bridge extends between the first air port and the second air port.

11. The method of operating a volatile material dispenser of claim 10, wherein the height of at least one of the air ports is between about 5% and about 20% of the chimney sheath height.

12. The method of operating a volatile material dispenser of claim 10, wherein the height of at least one of the air ports is between about 30% and about 50% of the chimney sheath height.

13. The method of operating a volatile material dispenser of claim 10, wherein at least one air port is disposed entirely within a bottom 50% of the chimney sheath height.

14. The method of operating a volatile material dispenser of claim 10, wherein the first air port and the second air port are disposed on opposing sides of the chimney sheath.

15. The method of operating a volatile material dispenser of claim 14, wherein the first air port and the second air port of the chimney sheath are rectangular.

16. A volatile material dispenser, comprising:
a housing;
a heater disposed within the housing; and
a refill having a container with a volatile material disposed therein and a wick in fluid communication with the volatile material and in thermal communication with the heater,
wherein the heater is configured to collapse when moving closer to the wick and expand when moving farther from the wick.

17. The volatile material dispenser of claim 16, wherein the dispenser includes an airflow enhancement mechanism in the form of a chimney sheath.

18. The volatile material dispenser of claim 16, wherein the heater is in the form of a tri-sectional heating element.

19. The volatile material dispenser of claim 18, wherein the sectional heating elements are connected with a wire and are in direct contact when in a fully collapsed configuration.

20. The volatile material dispenser of claim 19, wherein the heater in the fully collapsed configuration is oriented above the wick.
